(19) United States
(12) Patent Application Publication
(10) Pub. No.: US 2026/0055041 A1
(43) Pub. Date: Feb. 26, 2026

(54) A METHOD AND A SYSTEM FOR PRODUCING ETHER

(71) Applicant: Neste Oyj, Espoo (FI)

(72) Inventors: Matti KOSKINEN, Porvoo (FI); Hannu NOUSIAINEN, Porvoo (FI); Hanna-Maija HONKANEN, Porvoo (FI); Antti PYHÄLAHTI, Porvoo (FI); Antti KURKIJÄRVI, Porvoo (FI)

(73) Assignee: Neste Oyj, Espoo (FI)

(21) Appl. No.: 19/106,537

(22) PCT Filed: Aug. 7, 2023
(86) PCT No.: PCT/FI2023/050455

§ 371 Date: Feb. 25, 2025

(30) Foreign Application Priority Data
Aug. 26, 2022 (FI) 20225754

(51) Int. Cl. C07C 41/06; B01D 3/14; B01J 8/04; B01J 8/06; B01J 8/18; B01J 8/26; C07C 41/42

(52) U.S. Cl. CPC C07C 41/06; B01D 3/14; B01J 8/0492; B01J 8/0496; B01J 8/065; B01J 8/067; B01J 8/1827; B01J 8/1836; B01J 8/26; C07C 41/42; B01J 2208/00176; B01J 2208/065

(57) ABSTRACT

A system for producing ether includes a first etherification reactor system containing first catalyst and producing a first reaction effluent when supplied with alcohol and olefinic hydrocarbon feedstock, a second etherification reactor system containing second catalyst and producing a second reaction effluent when supplied with the first reaction effluent, and a distillation column receiving the second reaction effluent at a feed-point between the bottom and the top of the distillation column. The produced ether is taken out from the bottom of the distillation column. Volume of the first catalyst contributing the production of the first reaction effluent is at most 15% of total volume of the first catalyst and the second catalyst contributing the production of the second reaction effluent. The first etherification reactor system acts as a guard-bed reactor which removes unwanted components from the feed and thus protects the second etherification reactor system.

### FIELD OF THE DISCLOSURE

The disclosure relates generally to producing ethers such as tertiary alkyl ethers which can be used for example as components of motor fuels. The produced ethers may contain for example methyl t-butyl ether, ethyl t-butyl ether, t-amyl methyl ether, t-amyl ethyl ether, or mixtures thereof, and possibly heavier tertiary alkyl ethers. More particularly, the disclosure relates to a method for producing ethers and to a system for producing ethers.

### BACKGROUND

Tertiary alkyl ethers can be used as additives of gasoline to improve anti-knocking characteristics and to reduce harmful components in exhaust gases. The oxygen-containing ether group of tertiary alkyl ethers has been found advantageous to improve a combustion process of automotive engines and other similar engines. Examples of suitable tertiary alkyl ethers are: methyl t-butyl ether “MTBE”, ethyl t-butyl ether “ETBE”, t-amyl methyl ether “TAME”, t-amyl ethyl ether “TAEE”, t-hexyl methyl ether “THME”, and t-hexyl ethyl ether “THEE”. These ethers can be produced by etherification of olefinic hydrocarbon feedstock containing tertiary iso-olefins with monovalent aliphatic alcohols i.e. alkanols. The etherification reactions can be carried out in a fixed bed reactor, in a fluidized bed reactor, in an ebullated bed reactor, in a boiling-point reactor, in a tubular reactor, or in a catalytic distillation column.

In many cases, available data about the olefinic hydrocarbon feedstock as well as data about alcohol used in an etherification process lack detailed information about impurities present in the olefinic hydrocarbon feedstock and/or in the alcohol. Moreover, it is generally known that typical commercially available ethanol feedstocks contain compounds which have deteriorating and/or deactivating effects on etherification catalysts due to either fermentation or, more likely, due to storage and/or transportation. On the other hand, especially fluid catalytic cracking “FCC”-derived olefinic liquefied petroleum gas “LPG” contains basic nitrogen compounds which are direct catalyst poisons, as well as other basic material, ionic metal compounds and other deactivators. Pending on upstream processing facilities, a material stream from butadiene extraction may also contain basic nitrogen, in form of acetonitrile “ACN”, dimethylformamide “DMF”, and/or potentially harmful light compounds, especially in case of upsets. A common way to tackle these issues is to water wash the olefinic hydrocarbon feed or both the olefinic hydrocarbon feed and the alcohol feed. However, this approach requires an additional wash column with its auxiliary equipment, yields wastewater as continuous effluent, and saturates the hydrocarbon feed with water, which promotes by-product formation and corrosion in production equipment.

### SUMMARY

The following presents a simplified summary to provide a basic understanding of some embodiments of the invention. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In accordance with the invention, there is provided a new method for producing ether such as tertiary alkyl ether which may comprise for example methyl t-butyl ether “MTBE”, ethyl t-butyl ether “ETBE”, t-amyl methyl ether “TAME”, t-amyl ethyl ether “TAEE”, t-hexyl methyl ether “THME”, t-hexyl ethyl ether “THEE”, and/or mixtures thereof.

A method according to the invention comprises:
supplying alcohol and olefinic hydrocarbon feedstock, for example C₄₋₇ olefins, to a first etherification reactor system containing first catalyst to produce a first reaction effluent,
supplying the first reaction effluent from the first etherification reactor system to a second etherification reactor system containing second catalyst to produce a second reaction effluent,
supplying the second reaction effluent to a distillation column at a feed-point between a bottom and a top of the distillation column, and
taking out the produced ether from the bottom of the distillation column.

The volume of the first catalyst contributing the production of the first reaction effluent within the first etherification reactor system is at most 15% of total volume of the first catalyst and the second catalyst contributing the production of the second reaction effluent within the second etherification reactor system. The method comprises replacing, in response to deactivation of the first catalyst, the deactivated first catalyst with active catalyst material more often than replacing, in response to deactivation of the second catalyst, the deactivated second catalyst with active catalyst material.

The above-mentioned first etherification reactor system acts as a guard-bed reactor which removes unwanted components at least partly from the feed supplied to the second etherification reactor system, and thus the first etherification reactor system protects the second etherification reactor system from catalyst deactivation in addition to contributing the main process to produce ether. The catalyst of the first etherification reactor system needs to be changed more often than that of the second etherification reactor system, but costs of the first etherification reactor system, costs of the catalyst material to be changed, and costs for changing the catalyst material in the first etherification reactor system are smaller than corresponding capital and operating costs related to the water-wash approach mentioned in the background-section of this document.

In accordance with the invention, there is also provided a new system for producing ether. A system according to the invention comprises:
a first etherification reactor system containing first catalyst and configured to produce a first reaction effluent when being supplied with alcohol and olefinic hydrocarbon feedstock,
a second etherification reactor system containing second catalyst and configured to produce a second reaction effluent when being supplied with the first reaction effluent from the first etherification reactor system, and
a distillation column configured to receive the second reaction effluent at a feed-point between the bottom and the top of the distillation column, and comprising an outlet configured to remove the produced ether from the bottom of the distillation column,
   wherein the volume of the first catalyst contributing a production of the first reaction effluent within the first etherification reactor system is at most 15% of total volume of the first catalyst contributing the production of the first reaction effluent and the second catalyst contributing a production of the second reaction effluent within the second etherification reactor system.

Exemplifying and non-limiting embodiments of the invention are described in accompanied dependent claims.

Various exemplifying and non-limiting embodiments of the invention both to constructions and to methods of operation, together with additional objects and advantages thereof, will be best understood from the following description of specific exemplifying embodiments when read in connection with the accompanying drawings.

The verbs “to comprise” and “to include” are used in this document as open limitations that neither exclude nor require the existence of also un-recited features.

The features recited in the accompanied dependent claims are mutually freely combinable unless otherwise explicitly stated.

Furthermore, it is to be understood that the use of “a” or “an”, i.e. a singular form, throughout this document does as such not exclude a plurality.

### BRIEF DESCRIPTION OF THE FIGURES

Exemplifying and non-limiting embodiments of the invention and their advantages are explained in greater details below in the sense of examples and with reference to the accompanying drawings, in which:

FIG. 1 illustrates a system according to an exemplifying and non-limiting embodiment for producing ether,

FIG. 2 illustrates a system according to an exemplifying and non-limiting embodiment for producing ether,

FIG. 3 illustrates a system according to an exemplifying and non-limiting embodiment for producing ether, and

FIG. 4 shows a flowchart of a method according to an exemplifying and non-limiting embodiment for producing ether.

### DESCRIPTION OF EXEMPLIFYING EMBODIMENTS

The specific examples provided in the description below should not be construed as limiting the scope and/or the applicability of the accompanied claims. Lists and groups of examples provided in the description are not exhaustive unless otherwise explicitly stated.

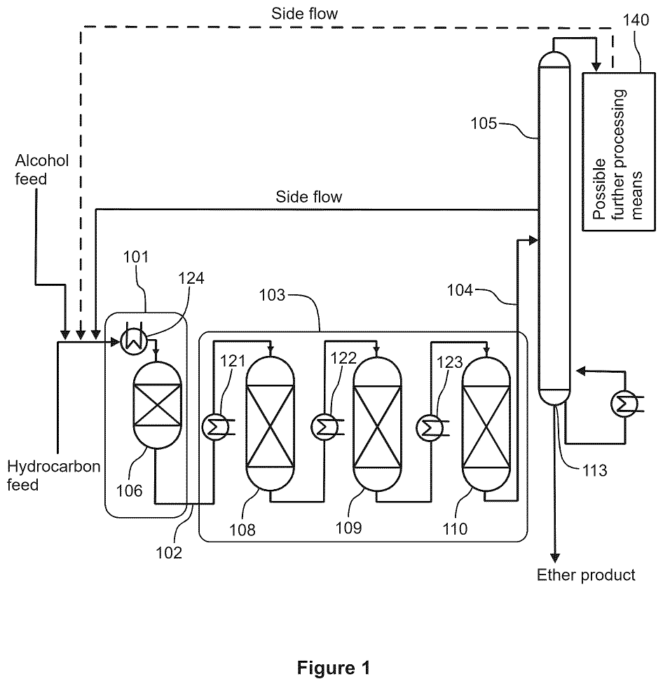

FIG. 1 illustrates a system according to an exemplifying and non-limiting embodiment for producing ether such as tertiary alkyl ether which may comprise for example methyl t-butyl ether “MTBE”, ethyl t-butyl ether “ETBE”, t-amyl methyl ether “TAME”, t-amyl ethyl ether “TAEE”, and t-hexyl methyl ether “THME”, t-hexyl ethyl ether “THEE”, and/or mixtures thereof. The exemplifying system illustrated in FIG. 1 comprises a first etherification reactor system 101 containing first catalyst in a reaction zone and configured to produce a first reaction effluent 102 when being supplied with alcohol and olefinic hydrocarbon feedstock. The olefinic hydrocarbon feedstock may comprise for example C₄₋₇ olefins comprising one or more of following compounds: isobutene, 2-methyl-1-butene, 2-methyl-2-butene, 2-methyl-1-pentene, 2-methyl-2-pentene, 2,3-dimethyl-1-butene, 2,3-dimethyl-2-butene, 2-ethyl-1-butene, 2-methyl-2-hexene, 2,3-dimethyl-1-pentene, 2,3-dimethyl-2-pentene, cis-3-methyl-2-pentene, trans-3-methyl-2-pentene, 2,4-dimethyl-1-pentene, 2-ethyl-1-pentene, 1-methyl cyclopentene, and 2-ethyl-2-pentene. The first catalyst may comprise for example cation exchange resin or zeolite. In this exemplifying system, the first etherification reactor system 101 comprises a reactor 106.

The exemplifying system illustrated in FIG. 1 comprises a second etherification reactor system 103 containing second catalyst in many reaction zones and configured to produce a second reaction effluent 104 when being supplied with the first reaction effluent 102 from the first etherification reactor system 101. The second catalyst may comprise for example cation exchange resin or zeolite. In the exemplifying system illustrated in FIG. 1, the second etherification reactor system 103 comprises three reactors 108, 109, and 110 which are connected in series.

The system comprises a distillation column 105 configured to receive the second reaction effluent 104 at a feed-point that is between the bottom and the top of the distillation column 105. The distillation column 105 comprises an outlet 113 configured to remove the produced ether from the bottom of the distillation column 105. The system may further comprise a side flow feed-back arrangement from the distillation column 105 to one or more suitable points in the first etherification reactor system 101 and/or in the second etherification reactor system 103.

The system may further comprise means 140 for further processing a material flow drawn out from the upper portion of the distillation column 105. The means 140 may comprise for example another distillation column whose operating pressure is higher than that of the distillation column 105. The operating pressure in the distillation column 105 can be typically in the range from 600 to 900 kPa, and in practice less than 1000 kPa. The operating pressure in the other distillation column can be typically in the range from 1500 to 2400 kPa, and in practice less than 3000 kPa. The means 140 may further comprise side flow feed-back arrangements from the other distillation column to one or more suitable points in the first etherification reactor system 101 and/or in the second etherification reactor system 103. For example, a side flow feed-back arrangement can be configured to draw a side flow from a suitable point of the other distillation column and to supply the side flow to the inlet of the first etherification reactor system 101. In FIG. 1, the side flow feed-back arrangement from the other distillation column is depicted with a dashed line.

The first etherification reactor system 101 acts as a guard-bed reactor which removes unwanted components from the feed materials supplied to the second etherification reactor system 103. Thus, the first etherification reactor system 101 protects the second etherification reactor system 103 from catalyst deactivation in addition to contributing the main process to produce ether. Therefore, the exemplifying system illustrated in FIG. 1 can be, and is, free from a water-wash column. The catalyst material of the first etherification reactor system 101 needs to be changed more often than that of the second etherification reactor system 103, but equipment costs of the first etherification reactor system 101, material costs of the catalyst material to be changed, and labor costs for changing the catalyst material in the first etherification reactor system 101 are smaller than for example corresponding capital and operating costs related to a water-wash column. The volume of the first catalyst contained by the reactor 106 of the first etherification reactor system 101 is at most 15% of the total volume of the first catalyst and the second catalyst that is contained by the reactors 108-110 of the second etherification reactor system 103. In other words, the volume of the first catalyst within the reactor 106 is at most about 18% of the volume of the second catalyst contained by the reactors 108-110. Advantageously, the volume of the first catalyst is from 10% to 15% of the total volume of the first and second catalysts. Each of the reactors 106 and 108-110 can be for example a fixed bed reactor, a fluidized bed reactor, an ebullated bed reactor, a boiling-point reactor, or a tubular reactor. In a fixed bed reactor, the reaction zone of the reactor is a catalyst bed. In a fluidized bed reactor, the reaction zone is a fluidized catalyst bed, in an ebullated bed reactor, the reaction zone is an ebullated catalyst bed, in a boiling-point reactor, the reaction zone is a fixed catalyst bed, and, in a tubular reactor, the reaction zone is formed by parallel tubes each containing catalyst material and each constituting a sub-zone.

The first etherification reactor system 101 further comprises a heat exchanger 124 for controlling temperature of the feed materials supplied to the reactor 106. Correspondingly, the second etherification reactor system 103 comprises heat exchangers 121, 122, and 123 for controlling temperatures of materials supplied to the reactors 108-110. In FIG. 1, temperature control means connected to the heat exchangers 121-124 are not shown. The input temperatures of the etherification reactors are typically in the range from 30° C. to 60° C., more advantageously from 40° C. to 45° C., and the output temperatures of the etherification reactors are advantageously at most from 70° C. to 80° C., and more advantageously below 70° C. The pressure within each etherification reactor is advantageously set to a value so that evaporation of C₄ hydrocarbons can either be avoided or controlled to maintain required temperature level in the reactor in case a boiling point reactor configuration is employed. The pressure within each etherification reactor is advantageously in the range from 1700 kPa to 1900 kPa.

An operational example of a system of the kind described above with reference to FIG. 1 is presented below. In this example case, the system is configured to produce ethyl t-butyl ether “ETBE”:

TABLE 1
Hydrocarbon feed 18150.6 kg/h

| Composition: | kmol/h | kg/h |
|---|---|---|
| Propene | 0.043 | 1.8 |
| Propane | 0.84 | 36.9 |
| 1,3-Butadiene | 1.18 | 63.5 |
| Isobutene | 161.2 | 9042.9 |
| Linear butenes | 72.71 | 4079.4 |
| Butanes | 84.74 | 4925.1 |
| Isopentane | 0.001 | 0.1 |
| Acetonitrile | 0.022 | 0.9 |

TABLE 2
Ethanol feed 7672.5 kg/h

| Composition: | kmol/h | kg/h |
|---|---|---|
| H₂O | 0.426 | 7.7 |
| MEOH | 0.191 | 6.1 |
| ETOH | 165.41 | 7620.3 |
| C₅ alcohol | 0.435 | 38.4 |

TABLE 3
Reactor sizes, i.e. volumes of catalyst

| | |
|---|---|
| Reactor 106: | 8 m³ |
| Reactor 108: | 12 m³ |
| Reactor 109: | 25 m³ |
| Reactor 110: | 25 m³ |
| Total: | 70 m³ |

Total conversion of isobutene: 97.65%

TABLE 4
ETBE product 16502 kg/h

| Composition: | kmol/h | kg/h |
|---|---|---|
| 1,3-Butadiene | 0.0002 | 0.006 |
| Isobutene | 0.0004 | 0.03 |
| Linear butenes | 0.0092 | 0.51 |
| Butanes | 0.0047 | 0.28 |
| Isopentane | 0.0012 | 0.09 |
| 2,4,4-trimethylpentene | 0.0004 | 0.05 |
| H₂O | 0.0601 | 1.1 |
| Tert-butyl alcohol | 0.4079 | 30.2 |
| MEOH | 0.00006 | 0.0 |
| MTBE | 0.1809 | 16.0 |
| ETOH | 8.4921 | 391.2 |
| Diethyl-ether | 0.0583 | 4.3 |
| Acetonitrile | 0.0 | 0.0 |
| C₅ alcohol | 0.4352 | 38.4 |
| ETBE | 156.7889 | 16020.1 |

A corresponding operational example of a system according to the prior art and comprising a water-wash column configured to wash the hydrocarbon feed is presented below. The main purpose of the water-wash column is to remove acetonitrile from the hydrocarbon feed. In this operational example, the diameter of the water-wash column is 1.2 m, the height of the water-wash column is 23 m, and the water-wash column has 40 trays. In a case of 50 weight-ppm acetonitrile concentration in hydrocarbon feed having a flow rate of 18150 kg/h, a need for washing water is about 11000 kg/h to achieve a target level 1 weight-ppm in the acetonitrile concentration.

In this exemplifying case according to the prior art, the etherification reactor system comprises three reactors which are connected in series and whose catalyst volumes are:

| | |
|---|---|
| The first reactor after the water-wash column | 20 m³ |
| The second reactor after the water-wash column: | 25 m³ |
| The third reactor after the water-wash column: | 25 m³ |
| Total: | 70 m³. |

The first reactor after the water-wash column is provided with a feedback circulation around the first reactor to limit temperature in the first reactor so that output temperature of the first reactor does not exceed 70° C. to avoid a decrease in selectivity to ETBE at higher temperatures. In the above-presented example according to an embodiment of the invention and comprising the guard-bed reactor, there is no need for a feedback circulation of the kind mentioned above.

TABLE 5
Hydrocarbon feed 18150.6 kg/h

| Composition: | kmol/h | kg/h |
|---|---|---|
| Propene | 0.043 | 1.8 |
| Propane | 0.84 | 36.9 |
| 1,3-Butadiene | 1.18 | 63.5 |
| Isobutene | 161.2 | 9042.9 |
| Linear butenes | 72.71 | 4079.4 |
| Butanes | 84.74 | 4925.1 |
| Isopentane | 0.001 | 0.1 |
| Acetonitrile | 0.022 | 0.9 |

TABLE 6
Ethanol feed 7669.8 kg/h

| Composition: | kmol/h | kg/h |
|---|---|---|
| H₂O | 0.426 | 7.7 |
| MEOH | 0.192 | 6.1 |
| ETOH | 165.35 | 7617.6 |
| C₅ alcohol | 0.435 | 38.4 |

Total conversion of isobutene: 97.64%

TABLE 7
ETBE product 16500 kg/h

| Composition: | kmol/h | kg/h |
|---|---|---|
| 1,3-Butadiene | 0.0001 | 0.006 |
| Isobutene | 0.0003 | 0.02 |
| Linear butenes | 0.0063 | 0.35 |
| Butanes | 0.0036 | 0.21 |
| Isopentane | 0.0012 | 0.09 |
| 2,4,4-trimethylpentene | 0.0005 | 0.06 |
| H₂O | 0.096 | 1.7 |
| Tert-butyl alcohol | 1.0024 | 40.9 |
| MEOH | 0.00006 | 0.0 |
| MTBE | 0.18 | 15.9 |
| ETOH | 8.60 | 396.3 |
| Diethyl-ether | 0.063 | 4.7 |
| Acetonitrile | 0.0 | 0.0 |
| C₅ alcohol | 0.44 | 38.4 |
| ETBE | 156.61 | 16001.6 |

As can be seen by comparing the above-presented examples, the system according to the embodiment of the invention provides substantially the same performance with the same total volume 70 m³ of the catalyst material but without a need for the water-wash column whose capital and operating costs are higher than those of the guard-bed reactor. Furthermore, the water washing produces wastewater about 11000 kg/h. The operational temperature of the water-wash column can be for example in the range from 15° C. to 50° C., more advantageously from 20° C. to 40° C., and the operational pressure of the water-wash column can be in the range from 800 kPa to 1000 kPa.

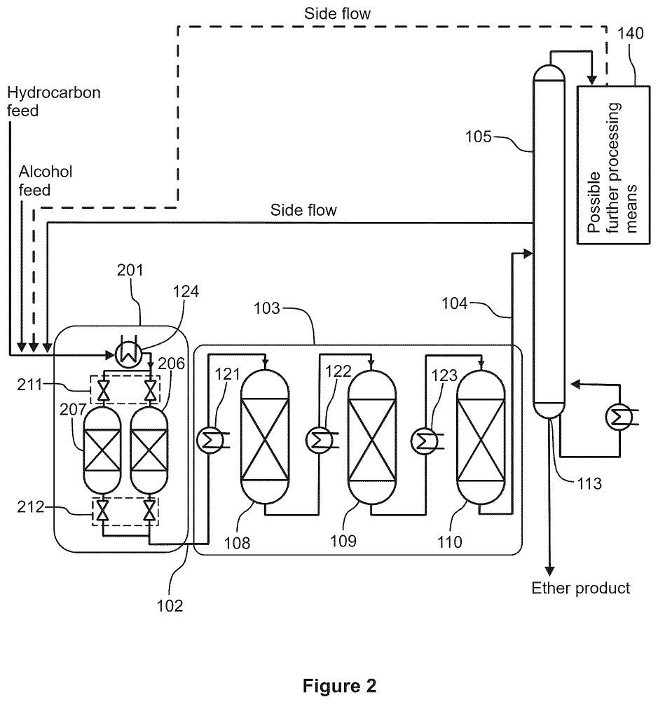

FIG. 2 illustrates a system according to an exemplifying and non-limiting embodiment for producing ether such as tertiary alkyl ether or a mixture of tertiary alkyl ethers. The system illustrated in FIG. 2 is otherwise like the system illustrated in FIG. 1, but the first etherification reactor system 201 comprises two reactors 206 and 207 connected in parallel and valves 211 and 212 for disconnecting each of the reactors 206 and 207 from the other reactor of the first etherification reactor system 201. The valves 211 and 212 enable each of the reactors 206 and 207 to be disconnected in response to a need to replace the catalyst material of the reactor under consideration with active catalyst material and to reconnect the disconnected reactor into operation, or to be ready for operation, after replacing its catalyst material. Catalyst materials of the reactors 206 and 207 can be changed in an alternating way so that one of the reactors 206 and 207 is in use in the main process for producing ether when catalyst material of the other one of the reactors 206 and 207 is being changed. Thus, there is no need to stop the main process during changing the catalyst material of the first etherification reactor system 201. The volume of the catalyst that contributes the production of the first reaction effluent 102 within the first etherification reactor system 201 is at most 15% of the total volume of the catalyst contributing the production of the first reaction effluent 102 and the catalyst contributing the production of the second reaction effluent 104 within the second etherification reactor system 103. Thus, in a case where only one of the reactors 206 and 207 is used at a time, the volume of the catalyst within the each of the reactors 206 and 207 is at most 15% of the above-mentioned total volume. Correspondingly, in a case where both the reactors 206 and 207 are used simultaneously, the volume of the catalyst within each of the reactors 206 and 207 is at most 7.5% of the above-mentioned total volume. Each of the reactors 206, 207, 108-110 can be for example a fixed bed reactor, a fluidized bed reactor, an ebullated bed reactor, a boiling-point reactor, or a tubular reactor.

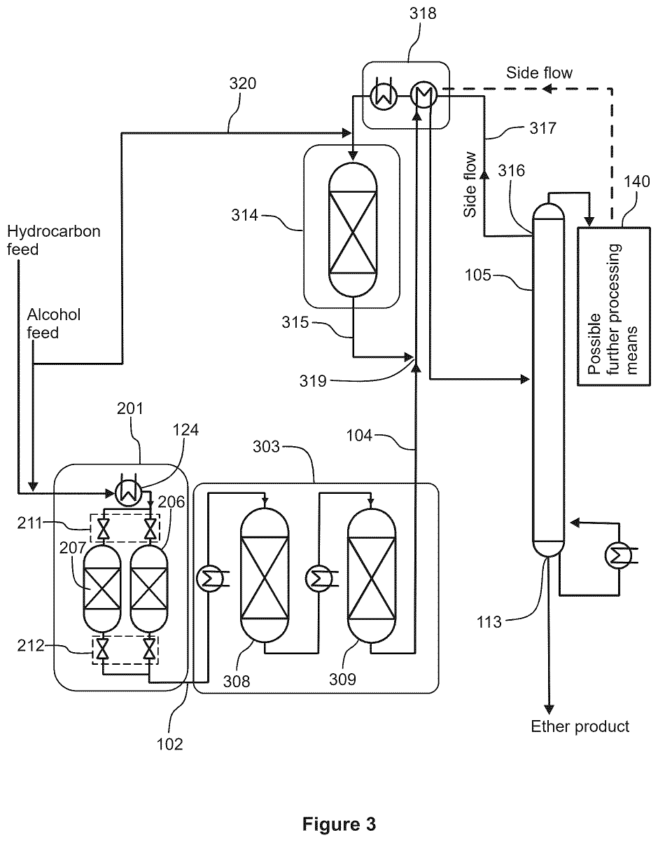

FIG. 3 illustrates a system according to an exemplifying and non-limiting embodiment for producing ether such as tertiary alkyl ether or a mixture of tertiary alkyl ethers. In this exemplifying system for producing ether, the first etherification reactor system 201 is like in the system illustrated in FIG. 2 and the second etherification reactor system 303 comprises two reactors 308 and 309 which are connected in series. The system illustrated in FIG. 3 comprises a side flow outlet 316 connected to a draw point between the feed-point of the distillation column 105 and the top of the distillation column. The side flow outlet 316 is configured to withdraw a side flow 317 from the distillation column 105. The side flow 317 is advantageously in vapor-phase. As an alternative, a side flow can be taken from another distillation column comprised by the means 140. This alternative is depicted with a dashed line in FIG. 3. The system comprises advantageously a heat exchanger system 318 configured to control temperature of the side flow 317. In a case the side flow 317 is a vapor-phase side flow, the heat exchanger system 318 is advantageously configured to condense the vapor-phase side flow into liquid. The system comprises a third etherification reactor system 314 containing third catalyst in a reaction zone and configured to receive the side flow 317 and to produce a third reaction effluent 315. The system comprises a piping 319 configured to mix the third reaction effluent 315 to the second reaction effluent 104 and to supply the mixture of the third and second reaction effluents to the feed-point of the distillation column 105. The system may further comprise an inlet piping 320 configured to supply a part of the alcohol feed to the third etherification reactor system 314 to improve the ether conversion in the third etherification reactor system 314. Each of the reactors shown in FIG. 3 can be for example a fixed bed reactor, a fluidized bed reactor, an ebullated bed reactor, a boiling-point reactor, or a tubular reactor.

The etherification reactions which take place in the first, second, and third etherification reactor systems 201, 303, and 314 are equilibrium reactions, and typically reaction rate slows down when ether concentration increases in a reactor under consideration. The side flow outlet 316 is at a such point in the distillation column 105 that the side flow 317 is substantially free from ethers or at least a relative concentration of ethers in the side flow 317 is low. Thus, the input feed to the third etherification reactor system 314 is substantially free from ethers and thereby the third etherification reactor system 314 provides more ether production for a given amount of catalyst material than the reactor 110 shown in FIGS. 1 and 2. Thus, thanks to the side flow recycle arrangement shown in FIG. 3, more ether can be produced with a same amount of catalyst material or, correspondingly, a same amount of ether can be produced with a smaller amount of catalyst material.

In the exemplifying system illustrated in FIG. 3, the heat exchanger system 318 is configured to transfer heat from the side flow 317 to the mixture of the second and third reaction effluents 104 and 315 prior to supplying the mixture of the second and third reaction effluents to the distillation column 105. This reduces the energy consumption of the system for producing ether.

An operational example of a system of the kind described above with reference to FIG. 3 is presented below. In this example case, the system is configured to produce ethyl t-butyl ether “ETBE”.

With the aid of simulations, it was found that the side flow 317 drawn from the distillation column 105 and returned to the inlet of the third etherification reactor system 314 has a beneficial impact on the amount of catalyst needed, i.e. the amount of catalyst can be reduced for a given amount of ETBE product. In this example, the side flow 317 is a vapor-phase side flow.

Table 8 shows a comparison between a system according to FIG. 3 for ethyl t-butyl ether “ETBE” production and a system according to FIG. 2 in which a side flow is drawn from the distillation column and returned to the inlet of the first etherification reactor system 201.

As can be seen by comparing the columns of Table 8 related to the system according to FIG. 2 and to the system according to FIG. 3, the system according to FIG. 3 provides substantially the same performance but with 45 m³ smaller total volume of the catalyst. The reactor systems acting as the guard-bed reactors, figure reference 201, are identical in these systems.

TABLE 8
Comparison of overall results for the system according
to FIG. 2 and the system according to FIG. 3.

| | System | System |
|---|---|---|
| | according to | according to |
| Entity | FIG. 2 | FIG. 3 |
| Outlet temperature of 1^{st} reactor, ° C. the | 68.8 | 68.8 |
| reactor 108 in FIG. 2 and the reactor | | |
| 308 in FIG. 3 | | |
| Outlet temperature of 2^{nd} reactor, ° C. the | 61.3 | 61.3 |
| reactor 109 in FIG. 2 and the reactor | | |
| 309 in FIG. 3 | | |
| Outlet temperature of 3^{rd} reactor, ° C. the | 41.5 | 44.9 |
| reactor 110 in FIG. 2 and the reactor of | | |
| the etherification reactor system 314 in | | |
| FIG. 3 | | |
| Catalyst volume of the 1^{st} reactor, m³ | 36 | 36 |
| the reactor 108 in FIG. 2 and the | | |
| reactor 308 in FIG. 3 | | |
| Catalyst volume of the 2^{nd} reactor, m³ | 90 | 90 |
| the reactor 109 in FIG. 2 and the | | |
| reactor 309 in FIG. 3 | | |
| Catalyst volume of the 3^{rd} reactor, m³ | 90 | 45 |
| reactor 110 in FIG. 2 and the reactor of | | |
| the etherification reactor system 314 in | | |
| FIG. 3 | | |
| Overall iso-butene conversion, % | 98.0 | 98.0 |
| Product ETBE flow, kg/h | 39225 | 39225 |
| Product ETBE content, % | 98.1 | 98.1 |
| Product ethanol content, % | 0.98 | 0.98 |
| Product water content, % | 0.03 | 0.03 |
| Product TBA content, % | 0.85 | 0.86 |
| Product DEE content, % | 0.02 | 0.009 |
| Product C₄ content, % | 0.000 | 0.000 |

The iso-butone conversion ratio, the product ETBE content weight-%, and the product ethanol content weight-% of the system according to FIG. 3 can be optimized by selecting a location of the side flow outlet 316 in the vertical direction of the distillation column 105 and by selecting a flow rate, kg/h, of the side flow 317 in an advantageous way.

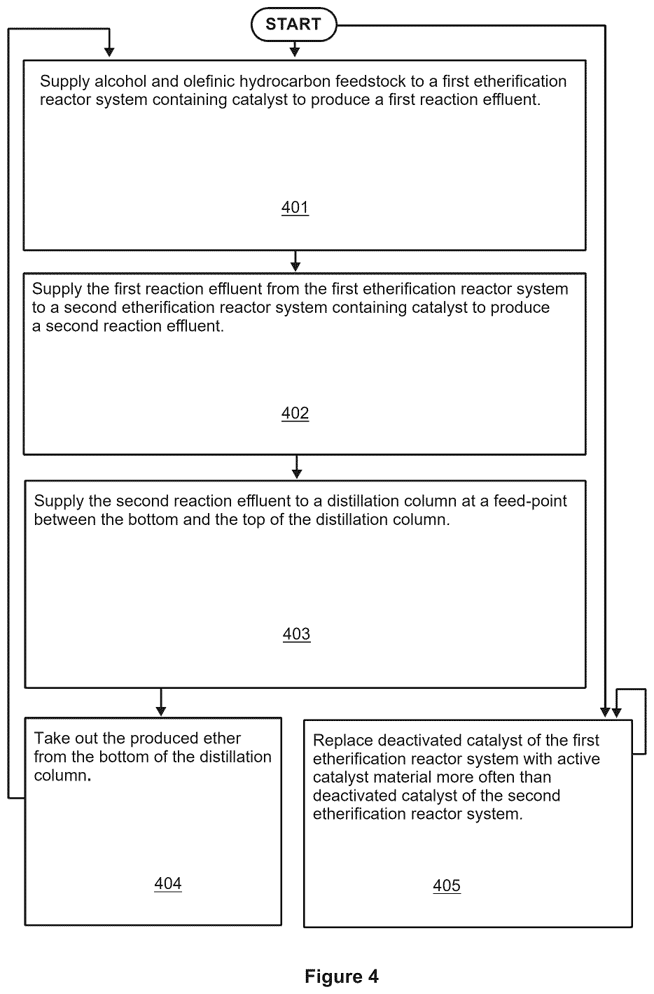

FIG. 4 shows a flowchart of a method according to an exemplifying and non-limiting embodiment for producing ether such as tertiary alkyl ether or a mixture of tertiary alkyl ethers. The method comprises the following actions:
action 401: supplying alcohol and olefinic hydrocarbon feedstock to a first etherification reactor system containing first catalyst in one or more reaction zones to produce a first reaction effluent,
action 402: supplying the first reaction effluent from the first etherification reactor system to a second etherification reactor system containing second catalyst in one or more reaction zones to produce a second reaction effluent, the volume of the first catalyst contributing a production of the first reaction effluent within the first etherification reactor system being at most 15% of the total volume of the first catalyst contributing the production of the first reaction effluent and the second catalyst contributing a production of the second reaction effluent within the second etherification reactor system,
action 403: supplying the second reaction effluent to a distillation column at a feed-point between the bottom and the top of the distillation column, and
action 404: taking out the produced ether from the bottom of the distillation column,

The above-mentioned method further comprises action 405: replacing the deactivated first catalyst of the first etherification reactor system with active catalyst material more often than replacing the deactivated second catalyst of the second etherification reactor system with active catalyst material. The first etherification reactor system acts as a guard-bed reactor which removes unwanted components from the feed materials supplied to the second etherification reactor system. Thus, the first etherification reactor system protects the second etherification reactor system from catalyst deactivation in addition to contributing the main process to produce ether. Therefore, catalyst of the first etherification reactor system needs to be changed more often than that of the second etherification reactor system.

The above-described method is advantageously a continuous process, which is illustrated by the arrow from the action 404 back to action 401 and by the arrow from the action 405 back to the action 405 i.e. back to itself.

In a method according to an exemplifying and non-limiting embodiment, the volume of the first catalyst within each reactor of the first etherification reactor system is from 10% to 15% of the total volume of the first and second catalysts contained by the first and second etherification reactor systems.

In a method according to an exemplifying and non-limiting embodiment, the first catalyst is the same catalyst material as the second catalyst.

In a method according to an exemplifying and non-limiting embodiment, the first catalyst comprises cation exchange resin or zeolite, and the second catalyst comprises cation exchange resin or zeolite.

In a method according to an exemplifying and non-limiting embodiment, the first etherification reactor system comprises at least two reactors connected in parallel and valves for disconnecting each of the reactors from the one or more other reactors of the first etherification reactor system, and the method comprises:
disconnecting, with the valves, one of the reactors of the first etherification reactor system in response to a situation which the first catalyst of the one of the reactors has been deactivated,
replacing the first catalyst of the disconnected reactor with active catalyst material, and
reconnecting the disconnected reactor into operation, or to be ready for operation, after replacing its first catalyst.

A method according to an exemplifying and non-limiting embodiment comprises changing the first catalyst of the above-mentioned at least two reactors of the first etherification reactor system in an alternating way in accordance with a predetermined timing schedule.

In a method according to an exemplifying and non-limiting embodiment, the second etherification reactor system comprises two or more reactors connected in series.

A method according to an exemplifying and non-limiting embodiment comprises:
withdrawing from the distillation column a side flow from a draw point between the feed-point of the distillation column and the top of the distillation column,
supplying the side flow to a third etherification reactor system containing third catalyst in one or more reaction zones to produce a third reaction effluent, and
mixing the third reaction effluent to the second reaction effluent prior to supplying the mixture of the second and third reaction effluents to the distillation column.

In a method according to an exemplifying and non-limiting embodiment, the above-mentioned side flow is a vapor-phase side flow, and the method comprises condensing the vapor-phase side flow into liquid prior to supplying the side flow to the third etherification reactor system.

In a method according to an exemplifying and non-limiting embodiment, the first catalyst, the second catalyst, and the third catalyst are the same catalyst material.

In a method according to an exemplifying and non-limiting embodiment, alcohol is supplied to the third etherification reactor system in addition to the side flow.

A method according to an exemplifying and non-limiting embodiment comprises cooling down the above-mentioned side flow and warming up the mixture of the second and third reaction effluents to be supplied to the distillation column by transferring heat from the side flow to the mixture of the second and third reaction effluents with a heat-exchanger.

In a method according to an exemplifying and non-limiting embodiment, the olefinic hydrocarbon feedstock comprises C₄₋₇ olefins comprising one or more of following compounds: isobutene, 2-methyl-1-butene, 2-methyl-2-butene, 2-methyl-1-pentene, cis-3-methyl-2-pentene, trans-3-methyl-2-pentene, 2-methyl-2-pentene, 2,3-dimethyl-1-butene, 2,3-dimethyl-2-butene, 2-ethyl-1-butene, 2-methyl-2-hexene, 2,3-dimethyl-1-pentene, 1-methyl cyclopentene, 2,3-dimethyl-2-pentene, 2,4-dimethyl-1-pentene, 2-ethyl-1-pentene, and 2-ethyl-2-pentene.

The specific examples provided in the description given above should not be construed as limiting. Therefore, the invention is not limited merely to the exemplifying and non-limiting embodiments described above. Lists and groups of examples provided in the description are not exhaustive unless otherwise explicitly stated.

1. A method for producing ether, the method comprising:
supplying alcohol and olefinic hydrocarbon feedstock to a first etherification reactor system containing first catalyst to produce a first reaction effluent;
supplying the first reaction effluent from the first etherification reactor system to a second etherification reactor system containing a second catalyst to produce a second reaction effluent;
supplying a second reaction effluent to a distillation column at a feed-point between a bottom and a top of the distillation column; and
taking out a produced ether from the bottom of the distillation column,
wherein a volume of the first catalyst contributing to a production of the first reaction effluent within the first etherification reactor system is at most 15% of a total volume of the first catalyst contributing a production of the first reaction effluent and the second catalyst contributing to a production of the second reaction effluent within the second etherification reactor system; and
replacing, in response to deactivation of the first catalyst, the deactivated first catalyst with active catalyst material more often than replacing, in response to deactivation of the second catalyst, the deactivated second catalyst with active catalyst material.

2. A method according to claim 1, wherein the volume of the first catalyst is from 10% to 15% of the total volume of the first and second catalysts.

3. A method according to claim 1, wherein the first etherification reactor system includes at least two reactors connected in parallel, and valves for disconnecting each of the reactors from one or more other reactors of the first etherification reactor system, and the method comprises:
disconnecting, with the valves, one of the reactors of the first etherification reactor system in response to a situation in which the first catalyst of the one of the reactors has been deactivated;
replacing the first catalyst of the disconnected reactor with active catalyst material; and
reconnecting the disconnected reactor into operation or to be ready for operation after replacing its first catalyst.

4. A method according to claim 3, wherein the method comprises:
changing the first catalyst of the at least two reactors of the first etherification reactor system in an alternating way in accordance with a predetermined timing schedule.

5. A method according to claim 1, wherein the second etherification reactor system includes two or more reactors connected in series.

6. A method according to claim 1, wherein the method comprises:
withdrawing, from the distillation column, a side flow from a draw point between the feed-point of the distillation column and the top of the distillation column;
supplying the side flow to a third etherification reactor system containing third catalyst to produce a third reaction effluent; and
mixing the third reaction effluent to the second reaction effluent prior to supplying a mixture of the second and third reaction effluents to the distillation column.

7. A method according to claim 6, wherein the side flow is a vapor-phase side flow, and the method comprises:
condensing the vapor-phase side flow into liquid prior to supplying the side flow to the third etherification reactor system.

8. A method according to claim 6, comprising:
supplying alcohol to the third etherification reactor system in addition to the side flow.

9. A method according to claim 6, wherein the method comprises:
cooling down the side flow and warming up the mixture of the second and third reaction effluents to be supplied to the distillation column by transferring heat from the side flow to the mixture of the second and third reaction effluents with a heat exchanger.

10. A method according to claim 1, wherein the first catalyst is a same material as the second catalyst.

11. A method according to claim 1, wherein the ether is tertiary alkyl ether or a mixture of tertiary alkyl ethers.

12. A method according to claim 1, wherein the first catalyst includes:
cation exchange resin or zeolite, and the second catalyst includes:
cation exchange resin or zeolite.

13. A method according to claim 1, wherein the olefinic hydrocarbon feedstock comprises:
C4-7 olefins including at least one or more of a following: isobutene, 2-methyl-1-butene, 2-methyl-2-butene, 2-methyl-1-pentene, 2-methyl-2-pentene, 2,3-dimethyl-1-butene, 2,3-dimethyl-2-butene, 2-ethyl-1-butene, 2-methyl-2-hexene, 2,3-dimethyl-1-pentene, cis-3-methyl-2-pentene, trans-3-methyl-2-pentene, 2,3-dimethyl-2-pentene, 2,4-dimethyl-1-pentene, 2-ethyl-1-pentene, 1-methyl cyclopentene, and/or 2-ethyl-2-pentene.

14. A system for producing ether, the system comprising:
a first etherification reactor system containing a first catalyst and configured to produce a first reaction effluent when supplied with alcohol and olefinic hydrocarbon feedstock;
a second etherification reactor system containing a second catalyst and configured to produce a second reaction effluent when supplied with the first reaction effluent from the first etherification reactor system; and
a distillation column configured to receive the second reaction effluent at a feed-point between a bottom and a top of the distillation column, and including an outlet configured to remove produced ether from the bottom of the distillation column,
wherein a volume of the first catalyst contributing to a production of the first reaction effluent within the first etherification reactor system is at most 15% of a total volume of the first catalyst contributing a production of the first reaction effluent and the second catalyst contributing to a production of the second reaction effluent within the second etherification reactor system.

15. A system according to claim 14, wherein the volume of the first catalyst is from 10% to 15% of the total volume of the first and second catalysts.

16. A system according to claim 14 wherein the first etherification reactor system comprises:
at least two reactors connected in parallel, and valves for disconnecting each of the reactors from one or more other reactors of the first etherification reactor system, the valves enabling each of the reactors of the first etherification reactor system to be disconnected to replace the first catalyst of the reactor with active catalyst material and to reconnect the disconnected reactor into operation, or to be ready for operation, after replacing the first catalyst of the reactor.

17. A system according to claim 14, wherein the second etherification reactor system comprises:
two or more reactors connected in series.

18. A system according to claim 14, comprising:
a side flow outlet between the feed-point of the distillation column and the top of the distillation column, the side flow outlet being configured to withdraw a side flow from the distillation column;
a third etherification reactor system containing a third catalyst and configured to receive the side flow and to produce a third reaction effluent; and
a piping configured to mix the third reaction effluent to the second reaction effluent and to supply a mixture of the third and second reaction effluents to the feed-point of the distillation column.

19. A system according to claim 18, comprising:
an inlet piping configured to supply alcohol to the third etherification reactor system.

20. A system according to claim 18, comprising:
a heat exchanger system configured to transfer heat from the side flow to the mixture of the second and third reaction effluents to be supplied to the distillation column, and wherein the third etherification reactor system is configured to receive the side flow from the heat exchanger system.

21. A system according to claim 20, wherein the heat exchanger system is configured to condense the side flow from vapor into liquid.
